(19) United States
(12) Patent Application Publication
(10) Pub. No.: US 2010/0184844 A1
(43) Pub. Date: Jul. 22, 2010

(54) Inhibition of Bacterial Protein Production by Polyvalent Oligonucleotide Modified Nanoparticle Conjugates

(75) Inventors: Chad A. Mirkin, Wilmette, IL (US); David A. Giljohann, Chicago, IL (US); Neema Navai, Chicago, IL (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: NORTHWESTERN UNIVERSITY, Evanston, IL (US)

(21) Appl. No.: 12/684,836

(22) Filed: Jan. 8, 2010

Related U.S. Application Data
(60) Provisional application No. 61/143,293, filed on Jan. 8, 2009.
(60) Provisional application No. 61/169,384, filed on Apr. 15, 2009.

(51) Int. Cl. A61K 48/00; A61P 31/00

(52) U.S. Cl. 514/44 R; 977/773; 977/906

(57) ABSTRACT

The present invention is directed to oligonucleotide-modified nanoparticle conjugates and methods of inhibiting bacterial protein production.

### CROSS REFERENCE TO RELATED APPLICATIONS

This application claims the priority benefit under 35 U.S.C. §119(e) of U.S. Provisional Application No. 61/143,293, filed Jan. 8, 2009, and U.S. Provisional Application No. 61/169,384, filed Apr. 15, 2009, which are incorporated by reference in their entirety.

### STATEMENT OF GOVERNMENT INTEREST

This invention was made with government support under Grant Number 5DP1 OD000285 awarded by the National Institutes of Health (NIH). The government has certain rights in the invention.

### FIELD OF THE INVENTION

The present invention is directed to oligonucleotide-modified nanoparticle conjugates and methods of inhibiting bacterial protein production.

### BACKGROUND OF THE INVENTION

Development of new agents to control bacterial proliferation is of paramount importance. Though molecular approaches to antibiotic agents have yielded meaningful results, current antibiotic treatments are becoming more limited as bacteria build resistance to antibiotics. Multiple classes of antibiotics exist targeting a myriad of bacterial functions. Though not an exhaustive list, some modalities include targeting of bacterial protein production (translational blockade, e.g., anti-ribosomal agents), bacterial cell wall integrity, and genome integrity (e.g., DNA gyrase). Nonetheless, the majority of these agents have been neutralized by bacterial evolution and the development of transmissible resistance, via conjugation, while the rest are expected to meet the same fate. In some cases bacterial resistance has jumped from one bacterial species to another. In addition, the current widespread use of antibiotics has lead to the emergence of “super strains” which resist most medical intervention. Therefore, new classes of drugs targeting bacteria are a research priority.

Polyvalent oligonucleotide nanoparticle conjugates have demonstrated significant ability for genetic regulation and detection strategies in eukaryotic systems. For genetic regulation, protein production has been blocked either by activation of RNA interference pathways, or by sequestration and/or degradation of mRNA in an antisense strategy. In the case of detection, mRNA binding to an oligonucleotide nanoparticle conjugate can be translated into a fluorescence signal. In mammalian cell culture systems, the nanoparticle conjugates are non-toxic and stable, have higher affinity for complementary targets, and are able to enter cells without transfection agents.

The use of oligonucleotides however, in bacteria, and in particular as a bactericide, has been of limited value. A limited number of agents have been developed, but their widespread use has never been adopted. While conceptually sound, the under use of this strategy is due to technical challenges (e.g., poor gene knockdown ability, inability to achieve intrabacteria) delivery, and stability of the oligonucleotide strands within the bacteria (i.e., nuclease resistance)).

### SUMMARY OF THE INVENTION

Described herein is an antibiotic composition comprising an oligonucleotide-modified nanoparticle and a carrier, wherein the oligonucleotide is sufficiently complementary to a target non-coding sequence of a prokaryotic gene to hybridize to the target sequence under conditions that allow hybridization. The antibiotic compositions described herein enters prokaryotic cells and regulates prokaryotic gene transcription and/or translation.

In some embodiments, an antibiotic composition is provided wherein hybridization to the prokaryotic gene inhibits growth of a prokaryotic cell. In another embodiment, an antibiotic composition is provided wherein hybridization of the oligonucleotide inhibits expression of a functional prokaryotic protein encoded by the prokaryotic gene. In one aspect, the antibiotic composition inhibits the expression of the functional prokaryotic protein by about 75% compared to a cell that is not contacted with the oligonucleotide-modified nanoparticle.

In a further embodiment, an antibiotic composition is provided wherein hybridization results in expression of a protein encoded by the prokaryotic gene with altered activity. In one aspect, an antibiotic composition is provided wherein the activity of the expressed gene product is reduced by about 10% compared to a cell that is not contacted with the oligonucleotide-modified nanoparticle. In an alternate aspect, an antibiotic composition is provided wherein the activity of the expressed gene product is increased by about 10% compared to a cell that is not contacted with the oligonucleotide-modified nanoparticle.

In another embodiment, an antibiotic composition is provided wherein hybridization of the oligonucleotide to the target sequence inhibits transcription of the prokaryotic gene. In another embodiment, an antibiotic composition is provided wherein hybridization of the oligonucleotide of the target sequence inhibits translation of a functional protein encoded by the prokaryotic gene.

The present disclosure further provides an antibiotic composition wherein hybridization of the oligonucleotide inhibits expression of a functional protein essential for prokaryotic cell growth. In various aspects, an antibiotic composition is provided wherein hybridization of the oligonucleotide inhibits expression of a functional protein essential for prokaryotic cell growth, the functional protein being essential for prokaryotic cell growth and selected from the group consisting of a gram-negative gene product, a gram-positive gene product, a cell cycle gene product, a gene product involved in DNA replication, a cell division gene product, a gene product involved in protein synthesis, a bacterial gyrase, and an acyl carrier gene product.

In another embodiment, an antibiotic composition is provided wherein the prokaryotic gene encodes a protein that confers a resistance to an antibiotic.

In some embodiments, an antibiotic composition is provided further comprising an antibiotic agent. In various aspects, an antibiotic composition is provided wherein the antibiotic agent is selected from the group consisting of Penicillin G, Methicillin, Nafcillin, Oxacillin, Cloxacillin, Dicloxacillin, Ampicillin, Amoxicillin, Ticarcillin, Carbenicillin, Mezlocillin, Azlocillin, Piperacillin, Imipenem, Aztreonam, Cephalothin, Cefaclor, Cefoxitin, Cefuroxime, Cefonicid, Cefmetazole, Cefotetan, Cefprozil, Loracarbef, Cefetamet, Cefoperazone, Cefotaxime, Ceftizoxime, Ceftriaxone, Ceftazidime, Cefepime, Cefixime, Cefpodoxime, Cefsulodin, Fleroxacin, Nalidixic acid, Norfloxacin, Ciprofloxacin, Ofloxacin, Enoxacin, Lomefloxacin, Cinoxacin, Doxycycline, Minocycline, Tetracycline, Amikacin, Gentamicin, Kanamycin, Netilmicin, Tobramycin, Streptomycin, Azithromycin, Clarithromycin, Erythromycin, Erythromycin estolate, Erythromycin ethyl succinate, Erythromycin glucoheptonate, Erythromycin lactobionate, Erythromycin stearate, Vancomycin, Teicoplanin, Chloramphenicol, Clindamycin, Trimethoprim, Sulfamethoxazole, Nitrofurantoin, Rifampin, Mupirocin, Metronidazole, Cephalexin, Roxithromycin, Co-amoxiclavuanate, combinations of Piperacillin and Tazobactam, and their various salts, acids, bases, and other derivatives.

In yet another embodiment, an antibiotic composition is provided wherein the oligonucleotide is sufficiently complementary to a sequence in a non-coding strand of the prokaryotic gene. In another embodiment, an antibiotic composition is provided wherein the oligonucleotide is sufficiently complementary to a sequence in a non-coding sequence of the prokaryotic gene to form a triple-stranded structure. In some aspects, an antibiotic composition is provided wherein hybridization forms a triple-stranded structure between the oligonucleotide and the non-coding sequence and a coding sequence complementary to the non-coding sequence. In further aspects, an antibiotic composition is provided wherein the oligonucleotide is sufficiently complementary to a sequence in the non-coding sequence of the prokaryotic gene to form a double-stranded structure between the oligonucleotide and the non-coding sequence. In some aspects, the non-coding sequence is a promoter sequence.

In some embodiments, an antibiotic composition is provided wherein the oligonucleotide hybridizes to a 3′ non-coding sequence. In further embodiments, an antibiotic composition is provided wherein the oligonucleotide hybridizes to a 5′ non-coding sequence.

The present disclosure also provides an antibiotic composition which hybridizes to the target sequence in vitro. In some embodiments, an antibiotic composition is provided which hybridizes to the target sequence in vivo.

Methods are herein provided for inhibiting production of a functional target gene product in a cell comprising the step of contacting the cell with the antibiotic composition of the present disclosure under conditions wherein hybridization results in inhibition of production of a functional protein encoded by the target gene.

In another embodiment, a method of treating a prokaryotic infection is provided comprising the step of administering to a cell a therapeutically effective amount of an antibiotic composition comprising the nanoparticle of the present disclosure.

Further provided herein is a kit comprising an antibiotic and the nanoparticle of the present disclosure.

### BRIEF DESCRIPTION OF THE DRAWINGS

FIG. 1 depicts a schematic of oligonucleotide gold nanoparticle conjugate blocking promoter complex binding (A) and full mRNA transcript formation (B) forming.

FIG. 2 depicts electron microscopy images of E. coli following conjugate treatment.

FIG. 3 depicts a summary of results for the inhibition of bacterial luciferase expression using nanoparticles. Nonsense denotes a sequence with no complementary region on the E. coli genome or transfected plasmid. Antisense denotes a sequence targeting luciferase. Relative luciferase activity is shown as percentages within the bars, normalized to renilla expression.

FIG. 4 depicts the duplex invasion scheme. A) Schematic of invasion of a duplex (fluorescein and adjacent dabcyl at terminus of duplex) by nanoparticle thereby releasing fluorescence signal. B) Results demonstrating increasing fluorescence with duplex invasion, both in short (20 base pair) duplexes and long (40 base pair) duplexes (Gray boxes represent nonsense sequences, Black boxes represent antisense sequences).

### DETAILED DESCRIPTION OF THE INVENTION

Provided herein is an antibiotic composition and methods of its use. In one aspect, the antibiotic composition comprises a nanoparticle modified to include an oligonucleotide, wherein the oligonucleotide is sufficiently complementary to a target non-coding sequence of a prokaryotic gene such that the oligonucleotide will hybridize to the target sequence under conditions that allow hybridization. Through this hybridization, the antibiotic composition inhibits growth of the target prokaryotic cell. In the target cell, in certain aspects, hybridization inhibits expression of a functional protein encoded by the targeted sequence. In various aspects, transcription, translation or both of a prokaryotic protein encoded by the targeted sequence is inhibited. The disclosure further provides a method of utilizing the antibiotic composition disclosed herein for inhibiting production of a target prokaryotic gene product in a cell comprising the step of contacting the cell with the antibiotic composition, wherein the oligonucleotide associated with the nanoparticle of the composition is sufficiently complementary to a target non-coding sequence of a bacterial gene under conditions that allow hybridization, and wherein hybridization results in inhibition of a functional prokaryotic gene product encoded by the target gene. It will be appreciated by those of ordinary skill in the art that inhibition of either transcription or translation, or both transcription and translation, of the target prokaryotic sequence results in the inhibition of production of a functional protein encoded by the target prokaryotic sequence.

Hybridization of an oligonucleotide-functionalized nanoparticle and a target prokaryotic sequence forms a “complex” as defined herein. As used herein, a “complex” is either a double-strand (or duplex) complex or a triple-strand (or triplex) complex. It is contemplated herein that a triplex complex and a duplex complex inhibit translation or transcription of a target bacterial prokaryotic acid.

As used herein, a “non-coding sequence” has a meaning accepted in the art. In general, non-coding sequence describes a polynucleotide sequence that does not contain codons for translation a protein encoded by the gene. In some aspects, a non-coding sequence is chromosomal. In some aspects, a non-coding sequence is extra-chromosomal. In one aspect, a non-coding sequence is complementary to all or part of the coding sequence of the gene. Non-coding sequences include regulatory elements such as promoters, enhancers, and silencers of expression. Examples of non-coding sequences are 5′ non-coding sequences and 3′ non-coding sequences. A “5′ non-coding sequence” refers to a polynucleotide sequence located 5′ (upstream) to the coding sequence. The 5′ non-coding sequence can be present in the fully processed mRNA upstream of the initiation codon and may affect processing of the primary transcript to mRNA, mRNA stability or translation efficiency. A “3′ non-coding sequence” refers to nucleotide sequences located 3′ (downstream) to a coding sequence and includes polyadenylation signal sequences and other sequences encoding signals capable of affecting mRNA processing or gene expression. The polyadenylation signal is usually characterized by its ability to affect the addition of polyadenylic acid sequences to the 3′ end of the mRNA precursor.

In one embodiment, a non-coding sequence comprises a promoter. A “promoter” is a polynucleotide sequence that directs the transcription of a structural gene. Typically, a promoter is located in the 5′ non-coding sequence of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements [DSEs; McGehee et al., Mol. Endocrinol. 7: 551 (1993)], cyclic AMP response elements (CREs), serum response elements [SREs; Treisman, Seminars in Cancer Biol. 1: 47 (1990)], glucocorticoid response elements (GREs), and binding sites for other transcription factors, such as CRE/ATF [O′Reilly et al., J. Biol. Chem. 267:19938 (1992)], AP2 [Ye et al., J. Biol. Chem. 269:25728 (1994)], SP1, cAMP response element binding protein [CREB; Loeken, Gene Expr. 3:253 (1993)] and octamer factors [see, in general, Watson et al., eds., Molecular Biology of the Gene, 4th ed. (The Benjamin/Cummings Publishing Company, Inc. 1987), and Lemaigre and Rousseau, Biochem. J. 303:1 (1994)]. If a promoter is an inducible promoter, then the rate of transcription increases in response to an inducing agent. In contrast, the rate of transcription is not regulated by an inducing agent if the promoter is a constitutive promoter. Repressible promoters are also known. A “core promoter” contains essential nucleotide sequences for promoter function, including the TATA box and start of transcription. By this definition, a core promoter may or may not have detectable activity in the absence of specific sequences that may enhance the activity or confer tissue specific activity.

In another embodiment, a non-coding sequence comprises a regulatory element. A “regulatory element” is a polynucleotide sequence that modulates the activity of a core promoter. For example, a regulatory element may contain a polynucleotide sequence that binds with cellular factors enabling transcription exclusively or preferentially in particular prokaryotes.

In another embodiment, a non-coding sequence comprises an enhancer. An “enhancer” is a type of regulatory element that can increase the efficiency of transcription, regardless of the distance or orientation of the enhancer relative to the start site of transcription.

It is noted here that, as used in this specification and the appended claims, the singular forms “a,” “an,” and “the” include plural reference unless the context clearly dictates otherwise.

It is to be noted that the terms “polynucleotide” and “oligonucleotide” are used interchangeably herein and have meanings accepted in the art.

It is further noted that the terms “attached”, “conjugated” and “functionalized” are also used interchangeably herein and refer to the association of an oligonucleotide with a nanoparticle.

“Hybridization” means an interaction between two or three strands of nucleic acids by hydrogen bonds in accordance with the rules of Watson-Crick DNA complementarity, Hoogstein binding, or other sequence-specific binding known in the art. Hybridization can be performed under different stringency conditions known in the art.

### Antibiotic Compositions

In some embodiments, the present disclosure provides antibiotic compositions comprising an oligonucleotide-modified nanoparticle and a carrier, wherein the oligonucleotide is sufficiently complementary to a target non-coding sequence of a prokaryotic gene that it will hybridize to the target sequence under conditions that allow hybridization. In various embodiments, the antibiotic compositions are formulated for administration in a therapeutically effective amount to a mammal in need thereof for the treatment of a prokaryotic cell infection. In some aspects, the mammal is a human.

In various embodiments, it is contemplated that hybridization of the oligonucleotide-modified nanoparticle to a prokaryotic gene inhibits (or prevents) the growth of a prokaryotic cell. Thus, the hybridization of the oligonucleotide-modified nanoparticle to a prokaryotic gene is contemplated to result in a bacteriostatic or bactericidal effect in aspects wherein the prokaryote is bacteria. In aspects wherein the hybridization occurs in vivo, the growth of the prokaryotic cell is inhibited by about 5% compared to the growth of the prokaryotic cell in the absence of contact with the oligonucleotide-modified nanoparticle. In various aspects, the growth of the prokaryotic cell is inhibited by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 2-fold, about 3-fold, about 4-fold, about 5-fold, about 6-fold, about 7-fold, about 8-fold, about 9-fold, about 10-fold, about 20-fold, about 50-fold or more compared to the growth of the prokaryotic cell in the absence of contact with the oligonucleotide-modified nanoparticle.

In aspects wherein the hybridization occurs in vitro, the growth of the prokaryotic cell is inhibited by about 5% compared to the growth of the prokaryotic cell in the absence of contact with the oligonucleotide-modified nanoparticle. In various aspects, the growth of the prokaryotic cell is inhibited by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 2-fold, about 3-fold, about 4-fold, about 5-fold, about 6-fold, about 7-fold, about 8-fold, about 9-fold, about 10-fold, about 20-fold, about 50-fold or more compared to the growth of the prokaryotic cell in the absence of contact with the oligonucleotide-modified nanoparticle.

Whether the inhibition is in vivo or in vitro, one of ordinary skill in the art can determine the level of inhibition of prokaryotic cell growth using routine techniques. For example, direct quantitation of the number of prokaryotic cells is performed by obtaining a set of samples (e.g., a bodily fluid in the case of in vivo inhibition or a liquid culture sample in the case of in vitro inhibition) wherein the samples are collected over a period of time, culturing the samples on solid growth-permissive media and counting the resultant number of prokaryotic cells that are able to grow. The number of prokaryotic cells at a later time point versus the number of prokaryotic cells at an earlier time point yields the percent inhibition of prokaryotic cell growth.

In some embodiments, hybridization of the oligonucleotide-modified nanoparticle to a prokaryotic gene inhibits expression of a functional prokaryotic protein encoded by the prokaryotic gene. A “functional prokaryotic protein” as used herein refers to a full length wild type protein encoded by a prokaryotic gene. In one aspect, the expression of the functional prokaryotic protein is inhibited by about 5% compared to a cell that is not contacted with the oligonucleotide-modified nanoparticle. In various aspects, the expression of the functional prokaryotic protein is inhibited by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 2-fold, about 3-fold, about 4-fold, about 5-fold, about 6-fold, about 7-fold, about 8-fold, about 9-fold, about 10-fold, about 20-fold, about 50-fold or more compared to a cell that is not contacted with the oligonucleotide-modified nanoparticle.

In related aspects, the hybridization of the oligonucleotide-modified nanoparticle to a prokaryotic gene inhibits expression of a functional protein essential for prokaryotic cell growth. In one aspect, the expression of the functional prokaryotic protein essential for prokaryotic cell growth is inhibited by about 5% compared to a cell that is not contacted with the oligonucleotide-modified nanoparticle. In various aspects, the expression of the functional prokaryotic protein essential for prokaryotic cell growth is inhibited by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 2-fold, about 3-fold, about 4-fold, about 5-fold, about 6-fold, about 7-fold, about 8-fold, about 9-fold, about 10-fold, about 20-fold, about 50-fold or more compared to a cell that is not contacted with the oligonucleotide-modified nanoparticle.

Prokaryotic proteins essential for growth include, but are not limited to, a gram-negative gene product, a gram-positive gene product, cell cycle gene product, a gene product involved in DNA replication, a cell division gene product, a gene product involved in protein synthesis, a bacterial gyrase, and an acyl carrier gene product. These classes are discussed in detail herein below.

The present disclosure also contemplates an antibiotic composition wherein hybridization to a target non-coding sequence of a prokaryotic gene results in expression of a protein encoded by the prokaryotic gene with altered activity. In one aspect, the activity of the protein encoded by the prokaryotic gene is reduced about 5% compared to the activity of the protein in a prokaryotic cell that is not contacted with the oligonucleotide-modified nanoparticle. In various aspects, activity of the prokaryotic protein is inhibited by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98% about 99% or about 100% compared to the activity of the protein in a prokaryotic cell that is not contacted with the oligonucleotide-modified nanoparticle. In another aspect, the activity of the protein encoded by the prokaryotic gene is increased about 5% compared to the activity of the protein in a prokaryotic cell that is not contacted with the oligonucleotide-modified nanoparticle. In various aspects, the expression of the prokaryotic protein is increased by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 2-fold, about 3-fold, about 4-fold, about 5-fold, about 6-fold, about 7-fold, about 8-fold, about 9-fold, about 10-fold, about 20-fold, about 50-fold or more compared to the activity of the protein in a prokaryotic cell that is not contacted with the oligonucleotide-modified nanoparticle.

The activity of the protein in a prokaryotic cell is increased or decreased as a function of several parameters including but not limited to the sequence of the oligonucleotide attached to the nanoparticle, the prokaryotic gene (thus and the protein encoded by the gene) that is targeted, and the size of the nanoparticle.

In various embodiments, it is contemplated that the antibiotic composition of the present disclosure inhibits transcription of the prokaryotic gene. In some embodiments, it is contemplated that the antibiotic composition of the present disclosure inhibits translation of the prokaryotic gene.

In some embodiments, the antibiotic composition hybridizes to a target non-coding sequence of a prokaryotic gene that confers a resistance to an antibiotic. These genes are known to those of ordinary skill in the art and are discussed, e.g., in Liu et al., Nucleic Acids Research 37: D443-D447, 2009 (incorporated herein by reference in its entirety). In some aspects, hybridization of the antibiotic composition to a target non-coding sequence of a prokaryotic gene that confers a resistance to an antibiotic results in increasing the susceptibility of the prokaryote to an antibiotic. In one aspect, the susceptibility of the prokaryote to the antibiotic is increased by about 5% compared to the susceptibility of the prokaryote that was not contacted with the antibiotic composition. In various aspects, the susceptibility of the prokaryote to the antibiotic is increased by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 2-fold, about 3-fold, about 4-fold, about 5-fold, about 6-fold, about 7-fold, about 8-fold, about 9-fold, about 10-fold, about 20-fold, about 50-fold or more compared to the susceptibility of the prokaryote that was not contacted with the antibiotic composition. Relative susceptibility to an antibiotic can be determined by those of ordinary skill in the art using routine techniques as described herein.

### Combination Therapy with Antibiotics

In some embodiments, the antibiotic composition comprising the oligonucleotide-modified nanoparticle conjugates are formulated for administration in combination with an antibiotic agent, each in a therapeutically effective amount.

The term “antibiotic agent” as used herein means any of a group of chemical substances having the capacity to inhibit the growth of, or to kill bacteria, and other microorganisms, used chiefly in the treatment of infectious diseases. See, e.g., U.S. Pat. No. 7,638,557 (incorporated by reference herein in its entirety). Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

### Dosing and Pharmaceutical Compositions

The term “therapeutically effective amount”, as used herein, refers to an amount of a composition sufficient to treat, ameliorate, or prevent the identified disease or condition, or to exhibit a detectable therapeutic, prophylactic, or inhibitory effect. The effect can be detected by, for example, an improvement in clinical condition, reduction in symptoms, or by an assay described herein. The precise effective amount for a subject will depend upon the subject's body weight, size, and health; the nature and extent of the condition; and the antibiotic composition or combination of compositions selected for administration. Therapeutically effective amounts for a given situation can be determined by routine experimentation that is within the skill and judgment of the clinician.

The antibiotic compositions described herein may be formulated in pharmaceutical compositions with a pharmaceutically acceptable excipient, carrier, or diluent. The compound or composition comprising the antibiotic composition can be administered by any route that permits treatment of the prokaryotic infection or condition. A preferred route of administration is oral administration. Additionally, the compound or composition comprising the antibiotic composition may be delivered to a patient using any standard route of administration, including parenterally, such as intravenously, intraperitoneally, intrapulmonary, subcutaneously or intramuscularly, intrathecally, transdermally, rectally, orally, nasally or by inhalation. Slow release formulations may also be prepared from the agents described herein in order to achieve a controlled release of the active agent in contact with the body fluids in the gastro intestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

Administration may take the form of single dose administration, or the compound of the embodiments can be administered over a period of time, either in divided doses or in a continuous-release formulation or administration method (e.g., a pump). However the compounds of the embodiments are administered to the subject, the amounts of compound administered and the route of administration chosen should be selected to permit efficacious treatment of the disease condition.

In an embodiment, the pharmaceutical compositions may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8. More particularly, the pharmaceutical compositions comprises in various aspects a therapeutically or prophylactically effective amount of at least one composition as described herein, together with one or more pharmaceutically acceptable excipients. As described herein, the pharmaceutical compositions may optionally comprise a combination of the compounds described herein.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

Additionally, the pharmaceutical compositions may be in the form of a sterile injectable preparation, such as a sterile injectable aqueous emulsion or oleaginous suspension. This emulsion or suspension may be formulated by a person of ordinary skill in the art using suitable dispersing or wetting agents and suspending agents. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids (e.g., oleic acid) may likewise be used in the preparation of injectables.

### Oligonucleotide Sequences and Inhibition of Prokaryotic Protein

In some aspects, the disclosure provides methods of targeting specific nucleic acids. Any type of prokaryotic nucleic acid may be targeted, and the methods may be used, e.g., for inhibition of production of a functional prokaryotic gene product. Examples of nucleic acids that can be targeted by the methods of the invention include but are not limited to genes and prokaryotic RNA or DNA.

For prokaryotic target nucleic acid, in various aspects, the nucleic acid is RNA transcribed from genomic DNA.

Methods for inhibiting production of a target prokaryotic protein in a cell provided include those wherein expression of the target gene product is inhibited by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or at least 100%, compared to gene product expression in the absence of an oligonucleotide-functionalized nanoparticle. In other words, methods provided embrace those which results in any degree of inhibition of expression of a target gene product.

The degree of inhibition is determined in vivo from, for example a body fluid sample of an individual in whom the target prokaryote is found and for which inhibition of a prokaryotic protein is desirable, or by imaging techniques in an individual in whom the target prokaryote is found and for which inhibition of a prokaryotic protein is desirable, well known in the art. Alternatively, the degree of inhibition is determined in vivo by quantitating the amount of a prokaryote that remains in cell culture or an organism compared to the amount of a prokaryote that was in cell culture or an organism at an earlier time point.

In embodiments where a triplex complex is formed, it is contemplated that a mutation is introduced to the prokaryotic genome. In these embodiments, the oligonucleotide-modified nanoparticle conjugate comprises the mutation and formation of a triplex complex initiates a recombination event between the oligonucleotide attached to the nanoparticle and a strand of the prokaryotic genome.

### Anti-Prokaryotic Oligonucleotides

The oligonucleotide of the present disclosure has a Tₘ, when hybridized with the target polynucleotide sequence, of at least about 45° C., typically between about 50° to 60° C., although the Tₘ may be higher, e.g., 65° C. The selection of prokaryotic target polynucleotide sequence, and prokaryotic mRNA target polynucleotide sequences are considered herein below.

In one embodiment, the oligonucleotides of the invention are designed to hybridize to a target prokaryotic sequence under physiological conditions, with a Tₘ substantially greater than 37° C., e.g., at least 45° C. and preferably 60° C.-80° C. The oligonucleotide is designed to have high binding affinity to the nucleic acid and, in one aspect, is 100% complementary to the target prokaryotic sequence, or it may include mismatches. Methods are provided in which the oligonucleotide is greater than 95% complementary to the target prokaryotic sequence, greater than 90% complementary to the target prokaryotic sequence, greater than 80% complementary to the target prokaryotic sequence, greater than 75% complementary to the target prokaryotic sequence, greater than 70% complementary to the target prokaryotic sequence, greater than 65% complementary to the target prokaryotic sequence, greater than 60% complementary to the target prokaryotic sequence, greater than 55% complementary to the target prokaryotic sequence, or greater than 50% complementary to the target prokaryotic sequence.

It will be understood that one of skill in the art may readily determine appropriate targets for oligonucleotide modified nanoparticle conjugates, and design and synthesize oligonucleotides using techniques known in the art. Targets can be identified by obtaining, e.g., the sequence of a target nucleic acid of interest (e.g. from GenBank) and aligning it with other nucleic acid sequences using, for example, the MacVector 6.0 program, a ClustalW algorithm, the BLOSUM 30 matrix, and default parameters, which include an open gap penalty of 10 and an extended gap penalty of 5.0 for nucleic acid alignments.

Any essential prokaryotic gene is contemplated as a target gene using the methods of the present disclosure. As described above, an essential prokaryotic gene for any prokaryotic species can be determined using a variety of methods including those described by Gerdes for E. coli [Gerdes et al., J Bacteriol. 185(19): 5673-84, 2003]. Many essential genes are conserved across the bacterial kingdom thereby providing additional guidance in target selection. Target gene sequences can be identified using readily available bioinformatics resources such as those maintained by the National Center for Biotechnology Information (NCBI). Complete reference genomic sequences for a large number of microbial species can be obtained and sequences for essential bacterial genes identified. Bacterial strains are also in one aspect obtained from the American Type Culture Collection (ATCC). Simple cell culture methods, using the appropriate culture medium and conditions for any given species, can be established to determine the antibacterial activity of oligonucleotide modified nanoparticle conjugates.

Oligonucleotide modified nanoparticle conjugates showing optimal activity are then tested in animal models, or veterinary animals, prior to use for treating human infection.

### Target Sequences for Cell-Division and Cell-Cycle Target Proteins

The oligonucleotides of the present disclosure are designed to hybridize to a sequence of a prokaryotic nucleic acid that encodes an essential prokaryotic gene. Exemplary genes include but are not limited to those required for cell division, cell cycle proteins, or genes required for lipid biosynthesis or nucleic acid replication. Any essential bacterial gene is a target once a gene's essentiality is determined. One approach to determining which genes in an organism are essential is to use genetic footprinting techniques as described [Gerdes et al., J Bacterial. 185(19): 5673-84, 2003, incorporated by reference herein in its entirety]. In this report, 620 E. coli genes were identified as essential and 3,126 genes as dispensable for growth under culture conditions for robust aerobic growth. Evolutionary context analysis demonstrated that a significant number of essential E. coli genes are preserved throughout the bacterial kingdom, especially the subset of genes for key cellular processes such as DNA replication, cell division and protein synthesis.

In various aspects, the present disclosure provides an oligonucleotide that is a nucleic acid sequence effective to stably and specifically bind to a target sequence which encodes an essential bacterial protein including the following: (1) a sequence specific to a particular strain of a given species of bacteria, such as a strain of E. coli associated with food poisoning, e.g., O157:H7 (see Table 1 of U.S. Patent Application Number 20080194463, incorporated by reference herein in its entirety); (2) a sequence common to two or more species of bacteria; (3) a sequence common to two related genera of bacteria (i.e., bacterial genera of similar phylogenetic origin); (4) a sequence generally conserved among Gram-negative bacteria; (5) generally conserved among Gram-positive bacteria; or (6) a consensus sequence for essential bacterial protein-encoding nucleic acid sequences in general.

In general, the target for modulation of gene expression using the methods of the present disclosure comprises a prokaryotic nucleic acid expressed during active prokaryotic growth or replication, such as an mRNA sequence transcribed from a gene of the cell division and cell wall synthesis (division cell wall or dcw) gene cluster, including, but not limited to, zipA, sulA, secA, dicA, dicB, dicC, dicF, ftsA, ftsI, ftsN, ftsK, ftsL, ftsQ, ftsW, ftsZ, murC, murD, murE, murF, murg, minC, minD, minE, mraY, mraW, mraZ, seqA and ddlB. See [Bramhill, Annu Rev Cell Dev Biol. 13: 395-424, 1997], and [Donachie, Annu Rev Microbiol. 47: 199-230, 1993], both of which are expressly incorporated by reference herein, for general reviews of bacterial cell division and the cell cycle of E. coli, respectively. Additional targets include genes involved in lipid biosynthesis (e.g. acpP) and replication (e.g. gyrA).

Cell division in E. coli involves coordinated invagination of all 3 layers of the cell envelope (cytoplasmic membrane, rigid peptidoglycan layer and outer membrane). Constriction of the septum severs the cell into two compartments and segregates the replicated DNA. At least 9 essential gene products participate in this process: ftsZ, ftsA, ftsQ, ftsL, ftsI, ftsN, ftsK, ftsW and zipA [Hale et al., J Bacteriol. 181(1): 167-76, 1999]. Contemplated protein targets are the three discussed below, and in particular, the GyrA and AcpP targets described below.

FtsZ, one of the earliest essential cell division genes in E. coli, is a soluble, tubulin-like GTPase that forms a membrane-associated ring at the division site of bacterial cells. The ring is thought to drive cell constriction, and appears to affect cell wall invagination. FtsZ binds directly to a novel integral inner membrane protein in E. coli called zipA, an essential component of the septal ring structure that mediates cell division in E. coli [Lutkenhaus et al., Annu Rev Biochem. 66: 93-116, 1997].

GyrA refers to subunit A of the bacterial gyrase enzyme, and the gene therefore. Bacterial gyrase is one of the bacterial DNA topoisomerases that control the level of supercoiling of DNA in cells and is required for DNA replication.

AcpP encodes acyl carrier protein, an essential cofactor in lipid biosynthesis. The fatty acid biosynthetic pathway requires that the heat stable cofactor acyl carrier protein binds intermediates in the pathway.

For each of these three proteins, Table 1 of U.S. Patent Application Number 20080194463 provides exemplary bacterial sequences which contain a target sequence for each of a number of important pathogenic bacteria. The gene sequences are derived from the GenBank Reference full genome sequence for each bacterial strain.

### Target Sequences for Prokaryotic 16S Ribosomal RNA

In one embodiment, the oligonucleotides of the invention are designed to hybridize to a sequence encoding a bacterial 16S rRNA nucleic acid sequence under physiological conditions, with a Tₘ substantially greater than 37° C., e.g., at least 45° C. and preferably 60° C.-80° C.

More particularly, the oligonucleotide has a sequence that is effective to stably and specifically bind to a target 16S rRNA gene sequence which has one or more of the following characteristics: (1) a sequence found in a double stranded sequence of a 16s rRNA, e.g., the peptidyl transferase center, the alpha-sarcin loop and the mRNA binding sequence of the 16S rRNA sequence; (2) a sequence found in a single stranded sequence of a bacterial 16s rRNA; (3) a sequence specific to a particular strain of a given species of bacteria, i.e., a strain of E. coli associated with food poisoning; (4) a sequence specific to a particular species of bacteria; (5) a sequence common to two or more species of bacteria; (6) a sequence common to two related genera of bacteria (i.e., bacterial genera of similar phylogenetic origin); (7) a sequence generally conserved among Gram-negative bacterial 16S rRNA sequences; (6) a sequence generally conserved among Gram-positive bacterial 16S rRNA sequences; or (7) a consensus sequence for bacterial 16S rRNA sequences in general.

Exemplary bacteria and associated GenBank Accession Nos. for 16S rRNA sequences are provided in Table 1 of U.S. Pat. No. 6,677,153, incorporated by reference herein in its entirety.

Escherichia coli (E. coli) is a Gram-negative bacterium that is part of the normal flora of the gastrointestinal tract. There are hundreds of strains of E. coli, most of which are harmless and live in the gastrointestinal tract of healthy humans and animals. Currently, there are four recognized classes of enterovirulent E. coli (the “EEC group”) that cause gastroenteritis in humans. Among these are the enteropathogenic (EPEC) strains and those whose virulence mechanism is related to the excretion of typical E. coli enterotoxins. Such strains of E. coli can cause various diseases including those associated with infection of the gastrointestinal tract and urinary tract, septicemia, pneumonia, and meningitis. Antibiotics are not effective against some strains and do not necessarily prevent recurrence of infection.

For example, E. coli strain O157:H7 is estimated to cause 10,000 to 20,000 cases of infection in the United States annually (Federa) Centers for Disease Control and Prevention). Hemorrhagic colitis is the name of the acute disease caused by E. coli O157:H7. Preschool children and the elderly are at the greatest risk of serious complications. E. coli strain O157:H7 was recently reported as the cause the death of four children who ate under-cooked hamburgers from a fast-food restaurant in the Pacific Northwest. [See, e.g., Jackson et al., Epidemiol. Infect. 120(1):17-20, 1998].

Exemplary sequences for enterovirulent E. coli strains include GenBank Accession Numbers X97542, AF074613, Y11275 and AJ007716.

Salmonella typhimurium, are Gram-negative bacteria that cause various conditions that range clinically from localized gastrointestinal infections, gastroenteritis (diarrhea, abdominal cramps, and fever) to enteric fevers (including typhoid fever) which are serious systemic illnesses. Salmonella infection also causes substantial losses of livestock.

Typical of Gram-negative bacilli, the cell wall of Salmonella spp. contains a complex lipopolysaccharide (LPS) structure that is liberated upon lysis of the cell and may function as an endotoxin, which contributes to the virulence of the organism.

Contaminated food is the major mode of transmission for non-typhoidal salmonella infection, due to the fact that Salmonella survive in meats and animal products that are not thoroughly cooked. The most common animal sources are chickens, turkeys, pigs, and cows; in addition to numerous other domestic and wild animals. The epidemiology of typhoid fever and other enteric fevers caused by Salmonella spp. is associated with water contaminated with human feces.

Vaccines are available for typhoid fever and are partially effective; however, no vaccines are available for non-typhoidal Salmonella infection. Non-typhoidal salmonellosis is controlled by hygienic slaughtering practices and thorough cooking and refrigeration of food. Antibiotics are indicated for systemic disease, and Ampicillin has been used with some success. However, in patients under treatment with excessive amounts of antibiotics, patients under treatment with immunosuppressive drugs, following gastric surgery, and in patients with hemolytic anemia, leukemia, lymphoma, or AIDS, Salmonella infection remains a medical problem.

Pseudomonas spp. are motile, Gram-negative rods which are clinically important because they are resistant to most antibiotics, and are a major cause of hospital acquired (nosocomial) infections. Infection is most common in immunocompromised individuals, burn victims, individuals on respirators, individuals with indwelling catheters, IV narcotic users and individual with chronic pulmonary disease (e.g., cystic fibrosis). Although infection is rare in healthy individuals, it can occur at many sites and lead to urinary tract infections, sepsis, pneumonia, pharyngitis, and numerous other problems, and treatment often fails with greater significant mortality.

Pseudomonas aeruginosa is a Gram-negative, aerobic, rod-shaped bacterium with unipolar motility. An opportunistic human pathogen, P. aeruginosa is also an opportunistic pathogen of plants. Like other Pseudomonads, P. aeruginosa secretes a variety of pigments. Definitive clinical identification of P. aeruginosa can include identifying the production of both pyocyanin and fluorescein as well as the organism's ability to grow at 42° C. P. aeruginosa is also capable of growth in diesel and jet fuel, for which it is known as a hydrocarbon utilizing microorganism (or “HUM bug”), causing microbial corrosion.

Vibrio cholera is a Gram-negative rod which infects humans and causes cholera, a disease spread by poor sanitation, resulting in contaminated water supplies. Vibrio cholerae can colonize the human small intestine, where it produces a toxin that disrupts ion transport across the mucosa, causing diarrhea and water loss. Individuals infected with Vibrio cholerae require rehydration either intravenously or orally with a solution containing electrolytes. The illness is generally self-limiting; however, death can occur from dehydration and loss of essential electrolytes. Antibiotics such as tetracycline have been demonstrated to shorten the course of the illness, and oral vaccines are currently under development.

Neisseria gonorrhoea is a Gram-negative coccus, which is the causative agent of the common sexually transmitted disease, gonorrhea. Neisseria gonorrhoea can vary its surface antigens, preventing development of immunity to reinfection. Nearly 750,000 cases of gonorrhea are reported annually in the United States, with an estimated 750,000 additional unreported cases annually, mostly among teenagers and young adults. Ampicillin, amoxicillin, or some type of penicillin used to be recommended for the treatment of gonorrhea. However, the incidence of penicillin-resistant gonorrhea is increasing, and new antibiotics given by injection, e.g., ceftriaxone or spectinomycin, are now used to treat most gonococcal infections.

Staphylococcus aureus is a Gram-positive coccus which normally colonizes the human nose and is sometimes found on the skin. Staphylococcus can cause bloodstream infections, pneumonia, and nosocomial infections. Staph. aureus can cause severe food poisoning, and many strains grow in food and produce exotoxins. Staphylococcus resistance to common antibiotics, e.g., vancomycin, has emerged in the United States and abroad as a major public health challenge both in community and hospital settings. Recently, a vancomycin-resistant Staph. aureus isolate has also been identified in Japan.

Mycobacterium tuberculosis is a Gram positive bacterium which is the causative agent of tuberculosis, a sometimes crippling and deadly disease. Tuberculosis is on the rise and globally and the leading cause of death from a single infectious disease (with a current death rate of three million people per year). It can affect several organs of the human body, including the brain, the kidneys and the bones, however, tuberculosis most commonly affects the lungs.

In the United States, approximately ten million individuals are infected with Mycobacterium tuberculosis, as indicated by positive skin tests, with approximately 26,000 new cases of active disease each year. The increase in tuberculosis (TB) cases has been associated with HIV/AIDS, homelessness, drug abuse and immigration of persons with active infections. Current treatment programs for drug-susceptible TB involve taking two or four drugs (e.g., isoniazid, rifampin, pyrazinamide, ethambutol or streptomycin), for a period of from six to nine months, because all of the TB germs cannot be destroyed by a single drug. In addition, the observation of drug-resistant and multiple drug resistant strains of Mycobacterium tuberculosis is on the rise.

Helicobacter pylori (H. pylori) is a micro-aerophilic, Gram-negative, slow-growing, flagellated organism with a spiral or S-shaped morphology which infects the lining of the stomach. H. pylori is a human gastric pathogen associated with chronic superficial gastritis, peptic ulcer disease, and chronic atrophic gastritis leading to gastric adenocarcinoma. H. pylori is one of the most common chronic bacterial infections in humans and is found in over 90% of patients with active gastritis. Current treatment includes triple drug therapy with bismuth, metronidazole, and either tetracycline or amoxicillin which eradicates H. pylori in most cases. Problems with triple therapy include patient compliance, side effects, and metronidazole resistance. Alternate regimens of dual therapy which show promise are amoxicillin plus metronidazole or omeprazole plus amoxicillin.

Streptococcus pneumoniae is a Gram-positive coccus and one of the most common causes of bacterial pneumonia as well as middle ear infections (otitis media) and meningitis. Each year in the United States, pneumococcal diseases account for approximately 50,000 cases of bacteremia; 3,000 cases of meningitis; 100,000-135,000 hospitalizations; and 7 million cases of otitis media. Pneumococcal infections cause an estimated 40,000 deaths annually in the United States. Children less than 2 years of age, adults over 65 years of age and persons of any age with underlying medical conditions, including, e.g., congestive heart disease, diabetes, emphysema, liver disease, sickle cell, HIV, and those living in special environments, e.g., nursing homes and long-term care facilities, at highest risk for infection.

Drug-resistant S. pneumoniae strains have become common in the United States, with many penicillin-resistant pneumococci also resistant to other antimicrobial drugs, such as erythromycin or trimethoprim-sulfamethoxazole.

Treponema pallidum is a spirochete which causes syphilis. T. pallidum is exclusively a pathogen which causes syphilis, yaws and non-venereal endemic syphilis or pinta. Treponema pallidum cannot be grown in vitro and does replicate in the absence of mammalian cells. The initial infection causes an ulcer at the site of infection; however, the bacteria move throughout the body, damaging many organs over time. In its late stages, untreated syphilis, although not contagious, can cause serious heart abnormalities, mental disorders, blindness, other neurologic problems, and death.

Syphilis is usually treated with penicillin, administered by injection. Other antibiotics are available for patients allergic to penicillin, or who do not respond to the usual doses of penicillin. In all stages of syphilis, proper treatment will cure the disease, but in late syphilis, damage already done to body organs cannot be reversed.

Chlamydia trachomatis is the most common bacterial sexually transmitted disease in the United States and it is estimated that 4 million new cases occur each year. The highest rates of infection are in 15 to 19 year olds. Chlamydia is a major cause of non-gonococcal urethritis (NGU), cervicitis, bacterial vaginitis, and pelvic inflammatory disease (PID). Chlamydia infections may have very mild symptoms or no symptoms at all; however, if left untreated Chlamydia infections can lead to serious damage to the reproductive organs, particularly in women. Antibiotics such as azithromycin, erythromycin, oflloxacin, amoxicillin or doxycycline are typically prescribed to treat Chlamydia infection.

Bartonella henselae Cat Scratch Fever (CSF) or cat scratch disease (CSD), is a disease of humans acquired through exposure to cats, caused by a Gram-negative rod originally named Rochalimaea henselae, and currently known as Bartonella henselae. Symptoms include fever and swollen lymph nodes and CSF is generally a relatively benign, self-limiting disease in people, however, infection with Bartonella henselae can produce distinct clinical symptoms in immunocompromised people, including, acute febrile illness with bacteremia, bacillary angiomatosis, peliosis hepatis, bacillary splenitis, and other chronic disease manifestations such as AIDS encephalopathy. The disease is treated with antibiotics, such as doxycycline, erythromycin, rifampin, penicillin, gentamycin, ceftriaxone, ciprofloxacin, and azithromycin.

Haemophilus influenzae (H. influenza) is a family of Gram-negative bacteria; six types of which are known, with most H. influenza-related disease caused by type B, or “HIB”. Until a vaccine for HIB was developed, HIB was a common causes of otitis media, sinus infections, bronchitis, the most common cause of meningitis, and a frequent culprit in cases of pneumonia, septic arthritis (joint infections), cellulitis (infections of soft tissues), and pericarditis (infections of the membrane surrounding the heart). The H. influenza type B bacterium is widespread in humans and usually lives in the throat and nose without causing illness. Unvaccinated children under age 5 are at risk for HIB disease. Meningitis and other serious infections caused by H. influenza infection can lead to brain damage or death.

Shigella dysenteriae (Shigella dys.) is a Gram-negative rod which causes dysentary. In the colon, the bacteria enter mucosal cells and divide within mucosal cells, resulting in an extensive inflammatory response. Shigella infection can cause severe diarrhea which may lead to dehydration and can be dangerous for the very young, very old or chronically ill. Shigella dys. forms a potent toxin (shiga toxin), which is cytotoxic, enterotoxic, neurotoxic and acts as a inhibitor of protein synthesis. Resistance to antibiotics such as ampicillin and TMP-SMX has developed, however, treatment with newer, more expensive antibiotics such as ciprofloxacin, norfloxacin and enoxacin, remains effective.

Listeria is a genus of Gram-positive, motile bacteria found in human and animal feces. Listeria monocytogenes causes such diseases as listeriosis, meningoencephalitis and meningitis. This organism is one of the leading causes of death from food-borne pathogens especially in pregnant women, newborns, the elderly, and immunocompromised individuals. It is found in environments such as decaying vegetable matter, sewage, water, and soil, and it can survive extremes of both temperatures and salt concentration making it an extremely dangerous food-born pathogen, especially on food that is not reheated. The bacterium can spread from the site of infection in the intestines to the central nervous system and the fetal-placental unit. Meningitis, gastroenteritis, and septicemia can result from infection. In cattle and sheep, listeria infection causes encephalitis and spontaneous abortion.

Proteus mirabilis is an enteric, Gram-negative commensal organism, distantly related to E. coli. It normally colonizes the human urethra, but is an opportunistic pathogen that is the leading cause of urinary tract infections in catheterized individuals. P. mirabilis has two exceptional characteristics: 1) it has very rapid motility, which manifests itself as a swarming phenomenon on culture plates; and 2) it produce urease, which gives it the ability to degrade urea and survive in the genitourinary tract.

Yersinia pestis is the causative agent of plague (bubonic and pulmonary) a devastating disease which has killed millions worldwide. The organism can be transmitted from rats to humans through the bite of an infected flea or from human-to-human through the air during widespread infection. Yersinia pestis is an extremely pathogenic organism that requires very few numbers in order to cause disease, and is often lethal if left untreated. The organism is enteroinvasive, and can survive and propagate in macrophages prior to spreading systemically throughout the host.

Bacillus anthracis is also known as anthrax. Humans become infected when they come into contact with a contaminated animal. Anthrax is not transmitted due to person-to-person contact. The three forms of the disease reflect the sites of infection which include cutaneous (skin), pulmonary (lung), and intestinal. Pulmonary and intestinal infections are often fatal if left untreated. Spores are taken up by macrophages and become internalized into phagolysozomes (membranous compartment) whereupon germination initiates. Bacteria are released into the bloodstream once the infected macrophage lyses whereupon they rapidly multiply, spreading throughout the circulatory and lymphatic systems, a process that results in septic shock, respiratory distress and organ failure. The spores of this pathogen have been used as a terror weapon.

Burkholderia mallei is a Gram-negative aerobic bacterium that causes Glanders, an infectious disease that occurs primarily in horses, mules, and donkeys. It is rarely associated with human infection and is more commonly seen in domesticated animals. This organism is similar to B. pseudomallei and is differentiated by being nonmotile. The pathogen is host-adapted and is not found in the environment outside of its host. Glanders is often fatal if not treated with antibiotics, and transmission can occur through the air, or more commonly when in contact with infected animals. Rapid-onset pneumonia, bacteremia (spread of the organism through the blood), pustules, and death are common outcomes during infection. The virulence mechanisms are not well understood, although a type III secretion system similar to the one from Salmonella typhimurium is necessary. No vaccine exists for this potentially dangerous organism which is thought to have potential as a biological terror agent. The genome of this organism carries a large number of insertion sequences as compared to the related Bukholderia pseudomallei (below), and a large number of simple sequence repeats that may function in antigenic variation of cell surface proteins.

Burkholderia pseudomallei is a Gram-negative bacterium that causes meliodosis in humans and animals. Meliodosis is a disease found in certain parts of Asia, Thailand, and Australia. B. pseudomallei is typically a soil organism and has been recovered from rice paddies and moist tropical soil, but as an opportunistic pathogen can cause disease in susceptible individuals such as those that suffer from diabetes mellitus. The organism can exist intracellularly, and causes pneumonia and bacteremia (spread of the bacterium through the bloodstream). The latency period can be extremely long, with infection preceding disease by decades, and treatment can take months of antibiotic use, with relapse a commonly observed phenomenon. Intercellular spread can occur via induction of actin polymerization at one pole of the cell, allowing movement through the cytoplasm and from cell-to-cell. This organism carries a number of small sequence repeats which may promoter antigenic variation, similar to what was found with the B. mallei genome.

Burkholderia cepacia is a Gram-negative bacterium composed of at least seven different sub-species, including Burkholderia multivorans, Burkholderia vietnamiensis, Burkholderia stabilis, Burkholderia cenocepacia and Burkholderia ambifaria. B. cepacia is an important human pathogen which most often causes pneumonia in people with underlying lung disease (such as cystic fibrosis or immune problems (such as (chronic granulomatous disease). B. cepacia is typically found in water and soil and can survive for prolonged periods in moist environments. Person-to-person spread has been documented; as a result, many hospitals, clinics, and camps for patients with cystic fibrosis have enacted strict isolation precautions B. cepacia. Individuals with the bacteria are often treated in a separate area than those without to limit spread. This is because infection with B. cepacia can lead to a rapid decline in lung function resulting in death. Diagnosis of B. cepacia involves isolation of the bacteria from sputum cultures. Treatment is difficult because B. cepacia is naturally resistant to many common antibiotics including aminoglycosides (such as tobramycin) and polymixin B. Treatment typically includes multiple antibiotics and may include ceflazidime, doxycycline, piperacillin, chloramphenicol, and co-trimoxazole.

Francisella tularensis was first noticed as the causative agent of a plague-like illness that affected squirrels in Tulare County in California in the early part of the 20th century by Edward Francis. The organism now bears his namesake. The disease is called tularemia and has been noted throughout recorded history. The organism can be transmitted from infected ticks or deerflies to a human, through infected meat, or via aerosol, and thus is a potential bioterrorism agent. It is an aquatic organism, and can be found living inside protozoans, similar to what is observed with Legionella. It has a high infectivity rate, and can invade phagocytic and nonphagocytic cells, multiplying rapidly. Once within a macrophage, the organism can escape the phagosome and live in the cytosol.

### Veterinary Applications

A healthy microflora in the gastrointestinal tract of livestock is of vital importance for health and corresponding production of associated food products. As with humans, the gastrointestinal tract of a healthy animal contains numerous types of bacteria (i.e., E. coli, Pseudomonas aeruginosa and Salmonella spp.), which live in ecological balance with one another. This balance may be disturbed by a change in diet, stress, or in response to antibiotic or other therapeutic treatment, resulting in bacterial diseases in the animals generally caused by bacteria such as Salmonella, Campylobacter, Enterococci, Tularemia and E. coli. Bacterial infection in these animals often necessitates therapeutic intervention, which has treatment costs as well being frequently associated with a decrease in productivity.

As a result, livestock are routinely treated with antibiotics to maintain the balance of flora in the gastrointestinal tract. The disadvantages of this approach are the development of antibiotic resistant bacteria and the carry over of such antibiotics and the resistant bacteria into resulting food products for human consumption.

### Nanoparticles

Nanoparticles are provided which are functionalized to have a polynucleotide attached thereto. The size, shape and chemical composition of the nanoparticles contribute to the properties of the resulting polynucleotide-functionalized nanoparticle. These properties include for example, optical properties, optoelectronic properties, electrochemical properties, electronic properties, stability in various solutions, magnetic properties, and pore and channel size variation. Mixtures of nanoparticles having different sizes, shapes and/or chemical compositions, as well as the use of nanoparticles having uniform sizes, shapes and chemical composition, and therefore a mixture of properties are contemplated. Examples of suitable particles include, without limitation, aggregate particles, isotropic (such as spherical particles), anisotropic particles (such as non-spherical rods, tetrahedral, and/or prisms) and core-shell particles, such as those described in U.S. Pat. No. 7,238,472 and International Publication No. WO 2003/08539, the disclosures of which are incorporated by reference in their entirety.

In one embodiment, the nanoparticle is metallic, and in various aspects, the nanoparticle is a colloidal metal. Thus, in various embodiments, nanoparticles of the invention include metal (including for example and without limitation, silver, gold, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials.

Also, as described in U.S. Patent Publication No 2003/0147966, nanoparticles of the invention include those that are available commercially, as well as those that are synthesized, e.g., produced from progressive nucleation in solution (e.g., by colloid reaction) or by various physical and chemical vapor deposition processes, such as sputter deposition. See, e.g., HaVashi, Vac. Sci. Technol. A5(4):1375-84 (1987); Hayashi, Physics Today, 44-60 (1987); MRS Bulletin, January 1990, 16-47. As further described in U.S. Patent Publication No 2003/0147966, nanoparticles contemplated are alternatively produced using HAuCl₄ and a citrate-reducing agent, using methods known in the art. See, e.g., Marinakos et al., Adv. Mater. 11:34-37 (1999); Marinakos et al., Chem. Mater. 10: 1214-19 (1998); Enustun & Turkevich, J. Am. Chem. Soc. 85: 3317 (1963).

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein.

### Oligonucleotides

The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases adenine (A), guanine (G), cytosine (C), thymine (T) and uracil (U). Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

A modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-sine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with a polynucleotide, or a modified form thereof, and a domain as defined herein, generally comprise a polynucleotide from about 5 nucleotides to about 100 nucleotides in length. More specifically, nanoparticles are functionalized with polynucleotide that are about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length are contemplated.

Polynucleotides contemplated for attachment to a nanoparticle include those which modulate expression of a gene product expressed from a target polynucleotide. Polynucleotides contemplated by the present disclosure include DNA, RNA and modified forms thereof as defined herein below. Accordingly, in various aspects and without limitation, polynucleotides which hybridize to a target polynucleotide and initiate a decrease in transcription or translation of the target polynucleotide, triple helix forming polynucleotides which hybridize to double-stranded polynucleotides and inhibit transcription, and ribozymes which hybridize to a target polynucleotide and inhibit translation, are contemplated.

In various aspects, if a specific polynucleotide is targeted, a single functionalized oligonucleotide-nanoparticle composition has the ability to bind to multiple copies of the same transcript. In one aspect, a nanoparticle is provided that is functionalized with identical polynucleotides, i.e., each polynucleotide has the same length and the same sequence. In other aspects, the nanoparticle is functionalized with two or more polynucleotides which are not identical, i.e., at least one of the attached polynucleotides differ from at least one other attached polynucleotide in that it has a different length and/or a different sequence. In aspects wherein different polynucleotides are attached to the nanoparticle, these different polynucleotides bind to the same single target polynucleotide but at different locations, or bind to different target polynucleotides which encode different gene products.

### Modified Oligonucleotides

As discussed above, modified oligonucleotides are contemplated for functionalizing nanoparticles. In various aspects, an oligonucleotide functionalized on a nanoparticle is completely modified or partially modified. Thus, in various aspects, one or more, or all, sugar and/or one or more or all internucleotide linkages of the nucleotide units in the polynucleotide are replaced with “non-naturally occurring” groups.

In one aspect, this embodiment contemplates a peptide nucleic acid (PNA). In PNA compounds, the sugar-backbone of a polynucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other linkages between nucleotides and unnatural nucleotides contemplated for the disclosed polynucleotides include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et. al., Current Opinion in Structural Biology 5:343-355 (1995) and Susan M. Freier and Karl-Heinz Altmann, Nucleic Acids Research, 25:4429-4443 (1997), the disclosures of which are incorporated herein by reference.

Specific examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide.”

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are polynucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated.

Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified polynucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In still other embodiments, polynucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In various forms, the linkage between two successive monomers in the oligo consists of 2 to 4, desirably 3, groups/atoms selected from —CH₂—, —O—, —S—, —NRH—, >C═O, >C═NRH, >C═S, Si(R″)₂, —SO—, —S(O)₂—, —P(O)₂—, —PO(BH₃)—, —P(O,S)—, —P(S)₂—, —PO(R″)—, —PO(OCH₃)—, and —PO(NHRH)—, where RH is selected from hydrogen and C1-4-alkyl, and R″ is selected from C1-6-alkyl and phenyl. Illustrative examples of such linkages are —CH₂—CH₂—CH₂—, —CH₂—CO—CH₂—, —CH₂—CHOH—CH₂—, —O—CH2-O—, —O—CH2-CH2-, —O—CH2-CH=(including R5 when used as a linkage to a succeeding monomer), —CH₂—CH₂—O—, —NRH—CH₂—CH₂—, —CH₂—CH₂—NRH—, —CH₂—NRH—CH₂—, —O—CH₂—CH₂—NRH—, —NRH—CO—O—, —NRH—CO—NRH—, —NRH—CS—NRH—, —NRH—C(═NRH)—NRH—, —NRH—CO—CH₂—NRH—O—CO—O—, —O—CO—CH₂—O—, —O—CH₂—CO—O—, CH₂—CO—NRH—, —O—CO—NRH—, —NRH—CO—CH₂—, —O—CH₂—CO—NRH—, —O—CH₂—CH₂—NRH—, —CH═N—O—, —CH₂—NRH—O—, —CH₂—O—N=(including R5 when used as a linkage to a succeeding monomer), —CH₂—O—NRH—, —CO—NRH—CH₂—, —CH₂—NRH—O—, —CH₂—NRH—CO—, —O—NRH—CH₂—, —O—NRH, —O—CH₂—S—, —S—CH₂—O—, —CH₂—CH₂—S—, —O—CH₂—CH₂—S—, —S—CH₂—CH=(including R5 when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂, —CH₂—SO₂—CH₂—, —O—SO—O—, —O—S(O)₂—O—, —O—S(O)₂—CH₂—, —O—S(O)₂—NRH—, —NRH—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHRN)—O—, —O—P(O)₂—NRH H—, —NRH—P(O)₂—O—, P(O,NRH)—O—, —CH₂—P(O)₂—O—, —O—P(O)₂—CH₂—, and —O—Si(R″)₂—O—; among which —CH₂—CO—NRH—, —CH₂—NRH—O—, —S—CH₂—O—, —O—P(O)₂—O—O—P(—O,S)—O—, —O—P(S)₂—O—, —NRH P(O)₂—O—, —O—P(O,NRH)—O—, —O—PO(R″)—O—, —O—PO(CH₃)—O—, and —O—PO(NHRN)—O—, where RH is selected form hydrogen and C₁₋₄-alkyl, and R″ is selected from C1-6-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of polynucleotides are described in detail in U.S. Patent Application No. 20040219565, the disclosure of which is incorporated by reference herein in its entirety.

Modified polynucleotides may also contain one or more substituted sugar moieties. In certain aspects, polynucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[CH₂)ₙO]ₘCH₃, O(CH2)ₙOCH₃, O(CH₂)NH₂, O(CH₂)ₙCH₃, O(CH₂)ₙONH₂, and O(CH₂)ₙON[(CH₂)CH₃]₂, where n and m are from 1 to about 10. Other polynucleotides comprise one of the following at the 2′ position: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a polynucleotide, or a group for improving the pharmacodynamic properties of a polynucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., 1995, Helv. Chim. Acta, 78: 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or T-DMAEOE), i.e., 2′—O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the polynucleotide, for example, at the 3′ position of the sugar on the 3′ terminal nucleotide or in 2′-5′ linked polynucleotides and the 5′ position of 5′ terminal nucleotide. Polynucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects a methylene (—CH₂—)n group bridging the 2′ oxygen atom and the 4′ carbon atom wherein n is 1 or 2. LNAs and preparation thereof are described in WO 98/39352 and WO 99/14226, the disclosures of which are incorporated herein by reference.

### Oligonucleotide Attachment to a Nanoparticle

Oligonucleotides contemplated for use in the methods include those bound to the nanoparticle through any means. Regardless of the means by which the oligonucleotide is attached to the nanoparticle, attachment in various aspects is effected through a 5′ linkage, a 3′ linkage, some type of internal linkage, or any combination of these attachments.

Methods of attachment are known to those of ordinary skill in the art and are described in US Publication No. 2009/0209629, which is incorporated by reference herein in its entirety. Methods of attaching RNA to a nanoparticle are generally described in PCT/US2009/65822, which is incorporated by reference herein in its entirety. Accordingly, in some embodiments, the disclosure contemplates that a polynucleotide attached to a nanoparticle is RNA.

In some aspects, nanoparticles with oligonucleotides attached thereto are provided wherein an oligonucleotide further comprising a domain is associated with the nanoparticle. In some aspects, the domain is a polythymidine sequence. In other aspects, the domain is a phosphate polymer (C3 residue).

In some embodiments, the oligonucleotide attached to a nanoparticle is DNA. When DNA is attached to the nanoparticle, the DNA is comprised of a sequence that is sufficiently complementary to a target sequence of a polynucleotide such that hybridization of the DNA oligonucleotide attached to a nanoparticle and the target polynucleotide takes place, thereby associating the target polynucleotide to the nanoparticle. The DNA in various aspects is single stranded or double-stranded, as long as the double-stranded molecule also includes a single strand sequence that hybridizes to a single strand sequence of the target polynucleotide. In some aspects, hybridization of the oligonucleotide functionalized on the nanoparticle can form a triplex structure with a double-stranded target polynucleotide. In another aspect, a triplex structure can be formed by hybridization of a double-stranded oligonucleotide functionalized on a nanoparticle to a single-stranded target polynucleotide.

### Spacers

In certain aspects, functionalized nanoparticles are contemplated which include those wherein an oligonucleotide and a domain are attached to the nanoparticle through a spacer. “Spacer” as used herein means a moiety that does not participate in modulating gene expression per se but which serves to increase distance between the nanoparticle and the functional oligonucleotide, or to increase distance between individual oligonucleotides when attached to the nanoparticle in multiple copies. Thus, spacers are contemplated being located between individual oligonucleotides in tandem, whether the oligonucleotides have the same sequence or have different sequences. In aspects of the invention where a domain is attached directly to a nanoparticle, the domain is optionally functionalized to the nanoparticle through a spacer. In aspects wherein domains in tandem are functionalized to a nanoparticle, spacers are optionally between some or all of the domain units in the tandem structure. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, an ethylglycol, or combinations thereof.

In certain aspects, the polynucleotide has a spacer through which it is covalently bound to the nanoparticles. These polynucleotides are the same polynucleotides as described above. As a result of the binding of the spacer to the nanoparticles, the polynucleotide is spaced away from the surface of the nanoparticles and is more accessible for hybridization with its target. In instances wherein the spacer is a polynucleotide, the length of the spacer in various embodiments at least about 10 nucleotides, 10-30 nucleotides, or even greater than 30 nucleotides. The spacer may have any sequence which does not interfere with the ability of the polynucleotides to become bound to the nanoparticles or to the target polynucleotide. The spacers should not have sequences complementary to each other or to that of the oligonucleotides, but may be all or in part complementary to the target polynucleotide. In certain aspects, the bases of the polynucleotide spacer are all adenines, all thymines, all cytidines, all guanines, all uracils, or all some other modified base.

### Surface Density

Nanoparticles as provided herein have a packing density of the polynucleotides on the surface of the nanoparticle that is, in various aspects, sufficient to result in cooperative behavior between nanoparticles and between polynucleotide strands on a single nanoparticle. In another aspect, the cooperative behavior between the nanoparticles increases the resistance of the polynucleotide to nuclease degradation. In yet another aspect, the uptake of nanoparticles by a cell is influenced by the density of polynucleotides associated with the nanoparticle. As described in PCT/US2008/65366, incorporated herein by reference in its entirety, a higher density of polynucleotides on the surface of a nanoparticle is associated with an increased uptake of nanoparticles by a cell.

A surface density adequate to make the nanoparticles stable and the conditions necessary to obtain it for a desired combination of nanoparticles and polynucleotides can be determined empirically. Generally, a surface density of at least 2 pmoles/cm² will be adequate to provide stable nanoparticle-oligonucleotide compositions. In some aspects, the surface density is at least 15 pmoles/cm². Methods are also provided wherein the polynucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

### EXAMPLES

### Example 1

### Preparation of Nanoparticles

Citrate-stabilized gold nanoparticles (from 1-250 nm) are prepared using published procedures [G. Frens, Nature Physical Science. 1973, 241, 20]. While a 13 and 5 nm size is used in this example, other examples include nanoparticles in size from 1 nm to 500 nm. Briefly, hydrogen tetrachloroaurate is reduced by treatment with citrate in refluxing water. The particle size and dispersity can be confirmed using transmission electron microscopy and uv/vis spectrophotometry. Thiolated oligonucleotides are synthesized using standard solid-phase phosphoramidite methodology [Pon, R. T. Solid-phase supports for oligonucleotide synthesis. Methods in Molecular Biology (Totowa, N.J., United States) (1993), 20 (Protocols for Oligonucleotides and Analogs), 465-496]. The thiol-modified oligonucleotides are next added to 13±1 and 5 nm gold colloids at a concentration of 3 nmol of oligonucleotide per 1 mL of 10 nM colloid and shaken overnight. After 12 hours, sodium dodecylsulphate (SDS) solution (10%) is added to the mixture to achieve a 0.1% SDS concentration, phosphate buffer (0.1 M; pH=7.4) is added to the mixture to achieve a 0.01 phosphate concentration, and sodium chloride solution (2.0 M) is added to the mixture to achieve a 0.1 M sodium chloride concentration. Six aliquots of sodium chloride solution (2.0 M) are then added to the mixture over an eight-hour period to achieve a final sodium chloride concentration of 0.3 M, and shaken overnight to complete the functionalization process. The solution is centrifuged (13,000 rpm, 20 min) and resuspended in sterile phosphate buffered saline three times to produce the purified conjugates.

### Example 2

### Oligonucleotide Modified Nanoparticle Conjugate Methods

Oligonucleotide design in this example includes two possible mechanisms of action. First, a sequence was designed using the published plasmid sequence that would preferentially hybridize to the sense strand of the promoter site for the Ampicillin resistance (AmpR) gene β-lactamase. This would sensitize the bacteria to ampicillin by taking advantage of the preferential hybridization of the conjugate (imparted by more favorable binding constant and/or intracellular concentration of the particles) to the promoter sequence of AmpR in the bacterial genome. This would prevent the promoter complex from binding to its target site and prevent transcription of the mRNA transcript (Amp resistance gene), therefore sensitizing the bacteria to ampicillin. The sequences used were 5′-AT TGT CTC ATG AGC GGA TAC ATA TTT GAA AAA AAA AAA A-SH-3′ (SEQ ID NO: 1) and 5′-AT TGT CTC ATG AGC GGA TAC AAA AAA AAA A-SH-3′ (SEQ ID NO: 2).

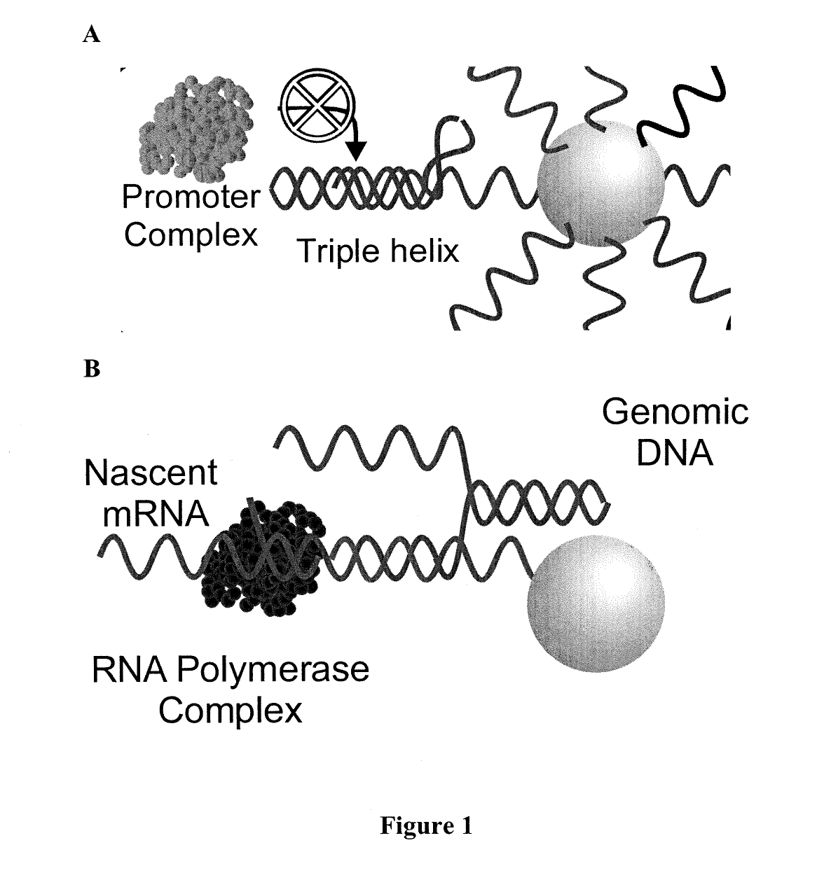

A second strategy would utilize a sequence designed to hybridize to an internal region of the AmpR gene. In doing so, this would prevent the completion of the full mRNA transcript. The downstream effect of this is to prevent complete transcription of functional mRNA transcript (Amp resistance gene) and therefore sensitize bacteria to ampicillin. For this strategy, a sense strand was chosen to hybridize to the target duplex DNA. The sequence for this was 5′-ACT TTT AAA GTT CTG CTA TAA AAA AAA AA-SH-3′ (SEQ ID NO: 3). A scheme for both strategies is presented in FIG. 1. Alternatively, one could use traditional antisense strategy to bind mRNA and prevent protein production, thus sensitizing the bacteria to antibiotics.

JM109 E. coli competent cells were transformed using an ampicillin containing plasmid (either psiCHECK 2, Promega or pScreen-iT, Invitrogen) according to published procedures (Promega and Invitrogen) and grown on antibiotic-containing (Amp) plates. A single colony was selected and grown in liquid culture with ampicillin for twelve hours. This culture was used to form a frozen (10% glycerol) stock for use in subsequent experiments.

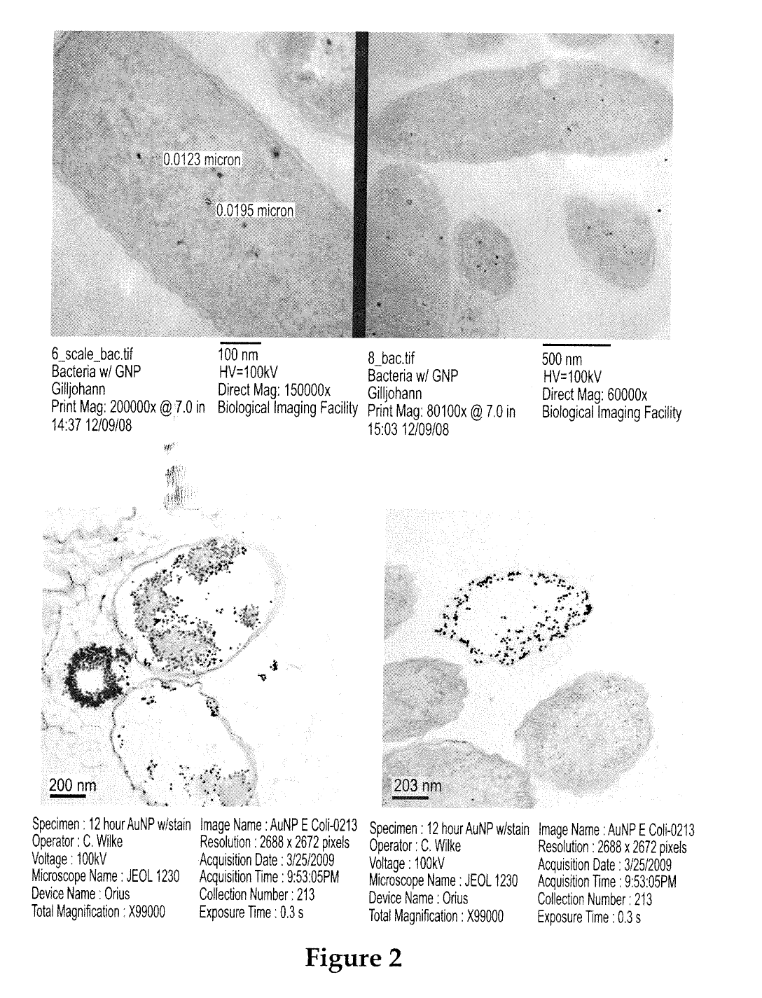

After thawing stocks of E. coli, a small volume was grown in liquid broth either with or without ampicillin as detailed below, and plated on corresponding LB plates. In one example, 54 of frozen bacterial broth was grown in 1 mL of LB broth with 30 nM particles for 5.5 hrs. From this 1 mL, 100 μL was plated and grown overnight. Bacterial entry was confirmed using transmission electron microscopy (FIG. 2).

After several hours of treatment with nanoparticles, a small volume of bacteria is plated on either ampicillin positive or ampicillin negative plates. The bacteria are grown on these plates for an additional twelve hours, and the number of colonies grown under each condition is evaluated. The results are summarized below in Table 1, below. A 66% inhibition of bacterial growth was obtained using this strategy. Routine optimization of conditions is expected to yield a 100% successful sensitization of bacteria.

TABLE 1

| | Trial | | | Expected |
|---|---|---|---|---|
| Growth Conditions | 1 | 2 | 3 | Growth |
| E. coli (−) | NA | NA | NA | (−) |
| Amp (−) | | | | |
| Nanoparticle (−) | | | | |
| E. coli (−) | (−) | (−) | (−) | (−) |
| Amp (+) | | | | |
| Nanoparticle (−) | | | | |
| E. coli (+) | NA | NA | NA | (+) |
| Amp (−) | | | | |
| NonsenseNP (+) | | | | |
| E. coli (+) | NA | NA | NA | (+) |
| Amp (+) | | | | |
| NonsenseNP (+) | | | | |
| E. coli (+) | (+) | (+) | (−) | (+) |
| Amp (−) | | | | |
| PromotorNP (+) | | | | |
| E. coli (+) | (−) | (−) | (−) | (−) |
| Amp (+) | | | | |
| PromotorNP (+) | | | | |
| E. coli (+) | (+) | (+) | (−) | (+) |
| Amp (−) | | | | |
| InternalNP (+) | | | | |
| E. coli (+) | (−) | (−) | (−) | (−) |
| Amp (+) | | | | |
| InternalNP (+) | | | | |
| E. coli (−) | (−) | (−) | (−) | (−) |
| Amp (−) | | | | |
| Nanoparticle (−) | | | | |
| E. coli (−) | (−) | (−) | (−) | (−) |
| Amp (+) | | | | |
| Nanoparticle (−) | | | | |
| E. coli (+) | (+) | (+) | (+) | (+) |
| Amp (−) | | | | |
| NonsenseNP (+) | | | | |
| E. coli (+) | (+) | (+) | (+) | (+) |
| Amp (+) | | | | |
| NonsenseNP (+) | | | | |
| E. coli (+) | (+) | (+) | (+) | (+) |
| Amp (−) | | | | |
| PromotorNP (+) | | | | |
| E. coli (+) | (−) | (−) | (+) | (−) |
| Amp (+) | | | | |
| PromotorNP (+) | | | | |
| E. coli (+) | (+) | (+) | (+) | (+) |
| Amp (−) | | | | |
| InternalNP (+) | | | | |
| E. coli (+) | (+) | (+) | (+) | (−) |
| Amp (+) | | | | |
| InternalNP (+) | | | | |

Protocol: 5 μL bacterial broth in 1 mL broth with 30 nM particles grown for 5.5 hrs. Plating of 100 μL and grown overnight.

### Example 3

### Oligonucleotide Modified Nanoparticle Conjugates Achieve Transcriptional Knockdown

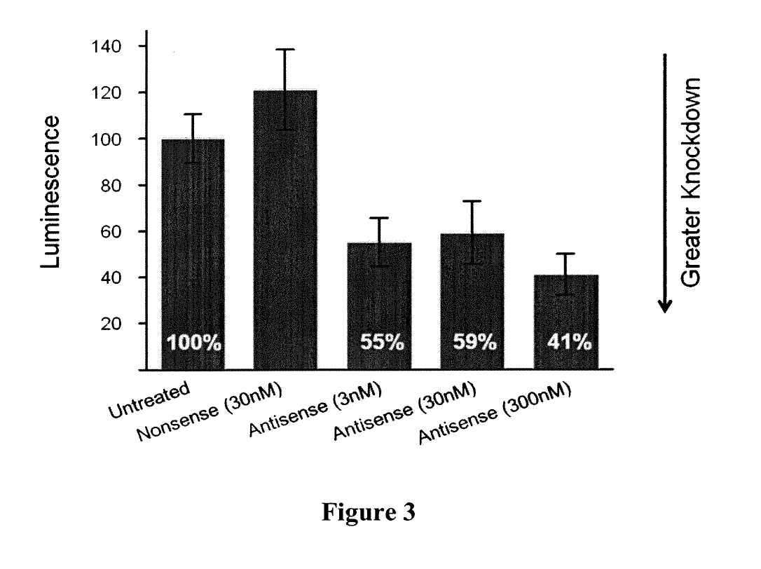

An additional strategy was employed to examine transcriptional knockdown in a plasmid derived Luciferase gene. This model was used to demonstrate site-selective gene knock down by differentiating Luciferase knockdown from a separate region on the plasmid encoding Renilla expression. To assay this effect the Dual-Luciferase Reporter Assay System (Promega) was used. The strategy employed for this model was to block formation of a full mRNA transcript of the luciferase gene. This results in diminution of luciferase signal in relation to renilla. The sequence used for this was 5′-CCC GAG CAA CGC AAA CGC AAA AAA AAA AA-SH-3′ (SEQ ID NO: 4). Alternatively, one could use a strategy similar to that used above to block the promoter complex from binding its target site. In this example, 5 nm particles were used. The resulting knockdown after 12 hours was 59% using 300 nM concentration of particles (p value=0.0004). These results demonstrate another method of achieving gene regulation at the transcriptional level. A summary of the data is shown in FIG. 3.

### Example 4

### Oligonucleotide Modified Nanoparticle Conjugate Blocking of Transcription

As a demonstration of these conjugates' ability to block transcription and subsequent protein production by hybridizing with double stranded genomic DNA, an in vitro transcription assay was conducted. Oligonucleotide functionalized gold nanoparticles were added in an in vitro transcription reaction (Promega) that contained double-stranded plasmid DNA encoding the luciferase gene. The oligonucleotide sequence targeted the sense strand of luciferase gene, thus could only block transcription and not translation. As a control, nanoparticle conjugates functionalized with non-complementary sequence was also used in an identical manner. The transcription reaction was allowed to proceed and luciferase activity was measured using a commercial kit (Promega). In the samples that contained nanoparticle conjugates that targeted the luciferase gene, a significant reduction in luciferase activity (>75%) was observed compared to control reactions that contained nanoparticle conjugates with non-complementary sequences.

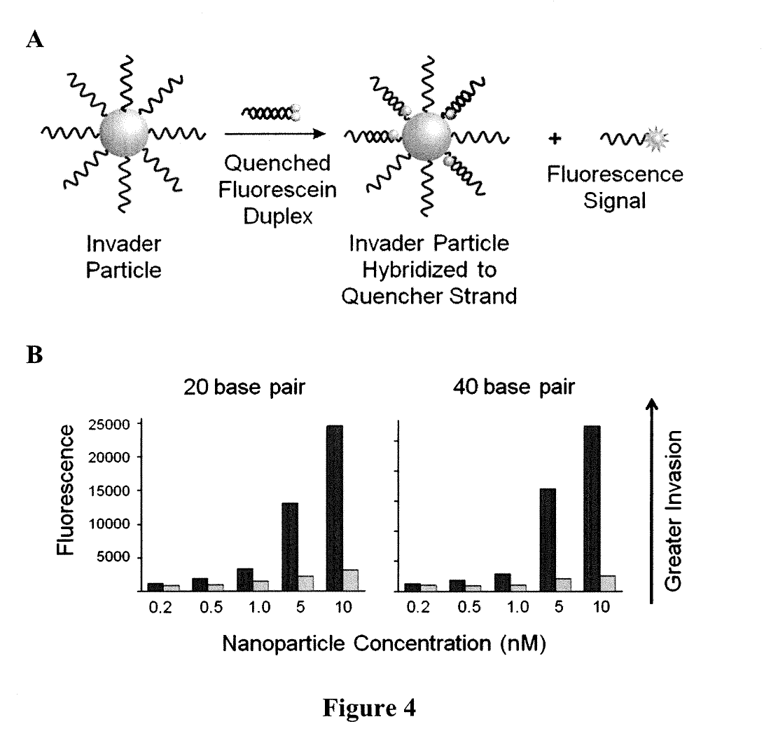

Additionally, to elucidate the underlying principle of knockdown, experiments were conducted in buffer to examine oligonucleotide gold nanoparticle conjugate invasion of a preformed duplex. A schematic and the resulting data are shown in FIGS. 4 (A and B). The particle may bind a preformed duplex (triplex formation). Alternatively, the particle may displace a preformed duplex via its higher binding constant for the target sequence. The particles are then centrifuged at 13,000 RPM, washed 3 times in PBS, and oxidized with KCN. Fluorescence of bound strands is measured. Without being bound by theory, this is hypothesized to result in the release of a fluorescein-capped oligonucleotide (antisense strand) and an increase in fluorescence signal. Prior to nanoparticle addition, a duplex with quencher (dabcyl, sense strand) and fluorophore (fluoroscein, antisense strand) are formed. Over a range of concentrations, sequence specificity for this strategy can be seen.

While the present invention has been described in terms of various embodiments and examples, it is understood that variations and improvements will occur to those skilled in the art. Therefore, only such limitations as appear in the claims should be placed on the invention.

1. An antibiotic composition comprising an oligonucleotide-modified nanoparticle, wherein the oligonucleotide is sufficiently complementary to a target non-coding sequence of a prokaryotic gene to hybridize to the target non-coding sequence under conditions that allow hybridization.

2. The antibiotic composition of claim 1 wherein hybridization to the prokaryotic gene inhibits growth of a prokaryotic cell.

3. The antibiotic composition of claim 1 wherein hybridization of the oligonucleotide inhibits expression of a functional prokaryotic protein encoded by the prokaryotic gene.

4. The antibiotic composition of claim 3 wherein expression of the functional prokaryotic protein is inhibited by about 75% compared to a cell that is not contacted with the oligonucleotide-modified nanoparticle.

5. The antibiotic composition of claim 1 wherein hybridization results in expression of a protein encoded by the prokaryotic gene with altered activity.

6. The antibiotic composition of claim 5 wherein the activity is reduced by about 10% compared to a cell that is not contacted with the oligonucleotide-modified nanoparticle.

7. The antibiotic composition of claim 1 wherein hybridization inhibits transcription of the prokaryotic gene.

8. The antibiotic composition of claim 1 wherein hybridization inhibits translation of a functional protein encoded by the prokaryotic gene.

9. The antibiotic composition of claim 1 wherein hybridization of the oligonucleotide inhibits expression of a functional protein essential for prokaryotic cell growth.

10. The antibiotic composition of claim 9 wherein hybridization of the oligonucleotide inhibits expression of a functional protein essential for prokaryotic cell growth, said functional protein essential for prokaryotic cell growth is selected from the group consisting of a gram-negative gene product, a gram-positive gene product, cell cycle gene product, a gene product involved in DNA replication, a cell division gene product, a gene product involved in protein synthesis, a bacterial gyrase, and an acyl carrier gene product.

11. The antibiotic composition of claim 1 wherein the prokaryotic gene encodes a protein that confers a resistance to an antibiotic.

12. The antibiotic composition of claim 1 further comprising an antibiotic agent selected from the group consisting of Penicillin G Methicillin Nafcillin Oxacillin Cloxacillin Dicloxacillin, Ampicillin Amoxicillin, Ticarcillin, Carbenicillin, Mezlocillin, Azlocillin, Piperacillin, Imipenem, Aztreonam, Cephalothin, Cefaclor, Cefoxitin, Cefuroxime, Cefonicid, Cefmetazole, Cefotetan, Cefprozil, Loracarbef, Cefetamet, Cefoperazone, Cefotaxime, Ceftizoxime, Ceftriaxone, Ceftazidime, Cefepime, Cefixime, Cefpodoxime, Cefsulodin, Fleroxacin, Nalidixic acid, Norfloxacin, Ciprofloxacin, Ofloxacin, Enoxacin, Lomefloxacin, Cinoxacin, Doxycycline, Minocycline, Tetracycline, Amikacin, Gentamicin, Kanamycin, Netilmicin, Tobramycin, Streptomycin, Azithromycin, Clarithromycin, Erythromycin, Erythromycin estolate Erythromycin ethyl succinate, Erythromycin glucoheptonate, Erythromycin lactobionate, Erythromycin stearate, Vancomycin, Teicoplanin, Chloramphenicol, Clindamycin, Trimethoprim, Sulfamethoxazole, Nitrofurantoin, Rifampin, Mupirocin, Metronidazole, Cephalexin, Roxithromycin, Co-amoxiclavuanate, combinations of Piperacillin and Tazobactam, and their various salts, acids, bases, and other derivatives.

13. (canceled)

14. The antibiotic composition of claim 1 wherein the oligonucleotide is sufficiently complementary to a sequence in a non-coding strand of the prokaryotic gene.

15. The antibiotic composition of claim 1 wherein the oligonucleotide is sufficiently complementary to a sequence in a non-coding sequence of the prokaryotic gene to form a triple-stranded structure.

16. The antibiotic composition of claim 1 wherein hybridization forms a triple-stranded structure between the oligonucleotide and the non-coding sequence and a coding sequence complementary to the non-coding sequence.

17. The antibiotic composition of claim 1 wherein the oligonucleotide is sufficiently complementary to a sequence in the non-coding sequence of the prokaryotic gene to form a double-stranded structure between the oligonucleotide and the non-coding sequence.

18. The antibiotic composition of claim 1 wherein the non-coding sequence is selected from the group consisting of a promoter sequence, a 3′ non-coding sequence, and a 5′ non-coding sequence.

19-20. (canceled)

21. The antibiotic composition of claim 1 which hybridizes to the target sequence in vitro and/or hybridizes to the target sequence in vivo.

22. (canceled)

23. A method of inhibiting production of a target gene product in a cell comprising the step of:
contacting the cell with the antibiotic composition of claim 1 under conditions wherein hybridization results in inhibition of production of a functional protein encoded by the target gene.

24. (canceled)

25. A kit comprising an antibiotic and the nanoparticle of claim 1.


SEQUENCE LISTING (4 sequences)

SEQ ID NO: 1 - DNA, 39 bp, Artificial Sequence
  Synthetic polynucleotide
attgtctcatgagcggatacatatttgaaaaaaaaaaaa

SEQ ID NO: 2 - DNA, 30 bp, Artificial Sequence
  Synthetic polynucleotide
attgtctcatgagcggatacaaaaaaaaaa

SEQ ID NO: 3 - DNA, 29 bp, Artificial Sequence
  Synthetic polynucleotide
acttttaaagttctgctataaaaaaaaaa

SEQ ID NO: 4 - DNA, 29 bp, Artificial Sequence
  Synthetic polynucleotide
cccgagcaacgcaaacgcaaaaaaaaaaa